(19) United States
(12) Patent Application Publication
(10) Pub. No.: US 2026/0198828 A1
(43) Pub. Date: Jul. 16, 2026

(54) PHYSIOLOGIC DETECTION APPARATUS AND SIGNAL DETECTION DEVICE

(71) Applicant: ZENTAN TECHNOLOGY CO., LTD., NEW TAIPEI CITY (TW)

(72) Inventors: TONG-PIE CHEN, NEW TAIPEI CITY (TW); YU-JU CHEN, NEW TAIPEI CITY (TW)

(21) Appl. No.: 19/023,926

(22) Filed: Jan. 16, 2025

(51) Int. Cl. A61B 5/256; A61B 5/00; A61B 5/27

(52) U.S. Cl. CPC A61B 5/256; A61B 5/27; A61B 5/6802; A61B 2560/0462; A61B 2562/168; A61B 2562/18; A61B 2562/227

(57) ABSTRACT

A signal detection device includes a wearable carrier, a waterproof layer disposed on the wearable carrier, a liquid storage layer disposed on the waterproof layer, a conductive fabric covering the liquid storage layer, an anti-slip layer disposed on the wearable carrier and spaced apart from the conductive fabric, and a signal transmission member. The conductive fabric has an extension portion that is not in contact with the liquid storage layer, and the signal transmission member pierces through and is fixed to the wearable carrier and the extension portion. The signal transmission member is spaced apart from the liquid storage layer by a predetermined spacing that is within a range from 5 mm to 12 mm. When the contact portion is in a moist state by being in contact with the liquid storage layer, the extension portion corresponding in position to the predetermined spacing is wetted through capillary action.

### FIELD OF THE DISCLOSURE

The present disclosure relates to a detection device, and more particularly to a physiologic detection apparatus and a signal detection device.

### BACKGROUND OF THE DISCLOSURE

When a conventional physiologic signal detection device is worn by a user and is configured to use a conductive fabric for detection, the conductive fabric needs to be maintained in a moist state for effectively transmitting signals from the user to a transmission member. However, if the transmission member encounters excessive liquid, a signal transmission of the transmission member can experience transmission errors. Moreover, when the conventional physiologic signal detection device encounters excessive liquid, the conventional physiologic signal detection device can easily slide or slip relative to the user, such as to cause detection errors.

### SUMMARY OF THE DISCLOSURE

In response to the above-referenced technical inadequacies, the present disclosure provides a physiologic detection apparatus and a signal detection device for effectively improving on the issues associated with conventional physiologic signal detection devices.

In order to solve the above-mentioned problems, one of the technical aspects adopted by the present disclosure is to provide a physiologic detection apparatus, which includes a signal detection device and a signal transceiver. The signal detection device includes a wearable carrier, a waterproof layer, a liquid storage layer, a conductive fabric, an anti-slip layer, and a signal transmission member. The wearable carrier has a first surface and a second surface that is opposite to the first surface. The waterproof layer is disposed on the first surface of the wearable carrier. The liquid storage layer is disposed on the waterproof layer so as to enable the wearable carrier to separate from the liquid storage layer through the waterproof layer. The conductive fabric includes a contact portion and an extension portion. The contact portion covers the liquid storage layer to enable the liquid storage layer to be positioned between the contact portion and the waterproof layer. The contact portion has a convex shape, and a maximum distance between the contact portion and the first surface is within a range from 150% to 250% of a thickness of the wearable carrier. The extension portion is connected to the contact portion and is not in contact with the liquid storage layer. The anti-slip layer is disposed on the first surface of the wearable carrier and is spaced apart from the conductive fabric. The signal transmission member pierces through and is fixed to the wearable carrier and the extension portion. The signal transmission member is spaced apart from the liquid storage layer by a predetermined spacing that is within a range from 5 mm to 12 mm. When the contact portion is in a moist state by being in contact with the liquid storage layer, the contact portion enables the extension portion corresponding in position to the predetermined spacing to be wetted through capillary action. The signal transceiver is detachably connected to the signal transmission member of the signal detection device. When the physiologic detection apparatus is worn by a user, the contact portion of the conductive fabric is configured to receive a physiologic signal from the user, and the extension portion and the signal transmission member are configured to transmit the physiologic signal from the contact portion to the signal transceiver.

In order to solve the above-mentioned problems, another one of the technical aspects adopted by the present disclosure is to provide a signal detection device, which includes a wearable carrier, a waterproof layer, a liquid storage layer, a conductive fabric, an anti-slip layer, and a signal transmission member. The wearable carrier has a first surface and a second surface that is opposite to the first surface. The waterproof layer is disposed on the first surface of the wearable carrier. The liquid storage layer is disposed on the waterproof layer so as to enable the wearable carrier to separate from the liquid storage layer through the waterproof layer. The conductive fabric includes a contact portion and an extension portion. The contact portion covers the liquid storage layer to enable the liquid storage layer to be positioned between the contact portion and the waterproof layer. The contact portion has a convex shape, and a maximum distance between the contact portion and the first surface is within a range from 150% to 250% of a thickness of the wearable carrier. The extension portion is connected to the contact portion and is not in contact with the liquid storage layer. The anti-slip layer is disposed on the first surface of the wearable carrier and is spaced apart from the conductive fabric. The signal transmission member pierces through and is fixed to the wearable carrier and the extension portion. The signal transmission member is spaced apart from the liquid storage layer by a predetermined spacing that is within a range from 5 mm to 12 mm. When the contact portion is in a moist state by being in contact with the liquid storage layer, the contact portion enables the extension portion corresponding in position to the predetermined spacing to be wetted through capillary action.

Therefore, the physiologic detection apparatus and the signal detection device in the present disclosure are each provided with a structural cooperation among the wearable carrier, the liquid storage layer, and the signal transmission member (e.g., the extension portion extends from the contact portion and is not in contact with the liquid storage layer, and the signal transmission member is spaced apart from the liquid storage layer by a predetermined spacing that is within a range from 5 mm to 12 mm), so that when the contact portion is in a moist state by being in contact with the liquid storage layer, the contact portion enables the extension portion corresponding in position to the predetermined spacing to be wetted through capillary action, thereby facilitating a signal transmission of the extension portion and effectively preventing a transmission quality of the signal transmission member from being affected by liquid.

Moreover, the physiologic detection apparatus and the signal detection device in the present disclosure are each provided with the contact portion having the convex shape and a specific size (e.g., the maximum distance between the contact portion and the first surface being within a range from 150% to 250% of the thickness of the wearable carrier), thereby enabling the contact portion to stably contact a to-be-detected portion of the user. Moreover, the wearable carrier of the signal detection device and the user have a gap therebetween through the contact portion, such that liquid (e.g., sweat) from the user can flow into the contact portion by passing through the gap for being stored in the liquid storage layer.

In addition, the physiologic detection apparatus and the signal detection device in the present disclosure are each provided with the anti-slip layer, so that when a lot of liquid exists between the wearable carrier and the user, the anti-slip layer is configured to effectively prevent the signal detection device from sliding relative to the user.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a physiologic detection apparatus according to a first embodiment of the present disclosure;

FIG. 2 is a schematic exploded view showing a part of the physiologic detection apparatus according to the first embodiment of the present disclosure;

FIG. 3 is a schematic planar view of the physiologic detection apparatus according to the first embodiment of the present disclosure;

FIG. 4 is a schematic enlarged view showing a part of FIG. 3;

FIG. 5 is a schematic cross-sectional view taken along line V-V of FIG. 4;

FIG. 6 is a schematic cross-sectional view taken along line VI-VI of FIG. 4;

FIG. 7 is a schematic planar view showing the part of FIG. 3 in another configuration;

FIG. 8 is a schematic enlarged view showing a part of the physiologic detection apparatus according to a second embodiment of the present disclosure;

FIG. 9 is a schematic enlarged view showing a part of the physiologic detection apparatus according to a third embodiment of the present disclosure; and

FIG. 10 is a schematic cross-sectional view taken along line X-X of FIG. 9.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

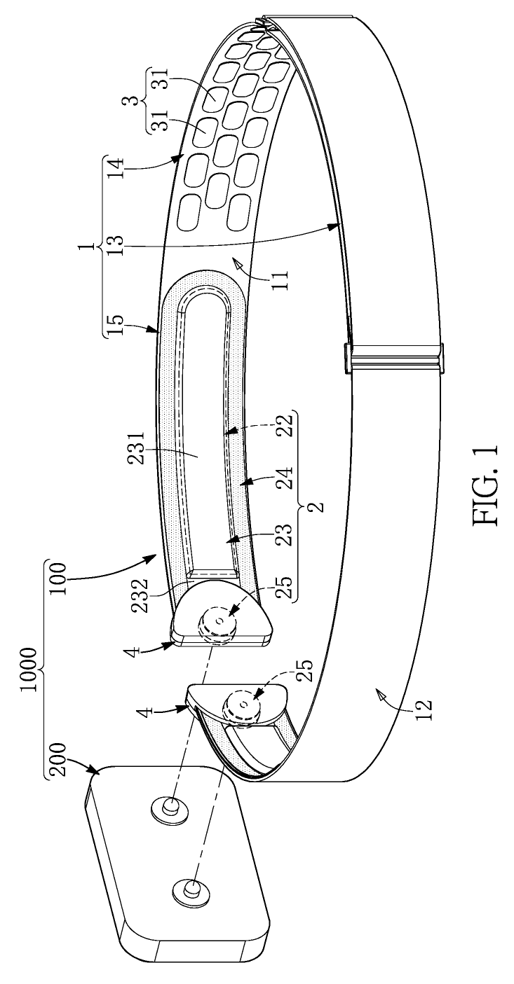

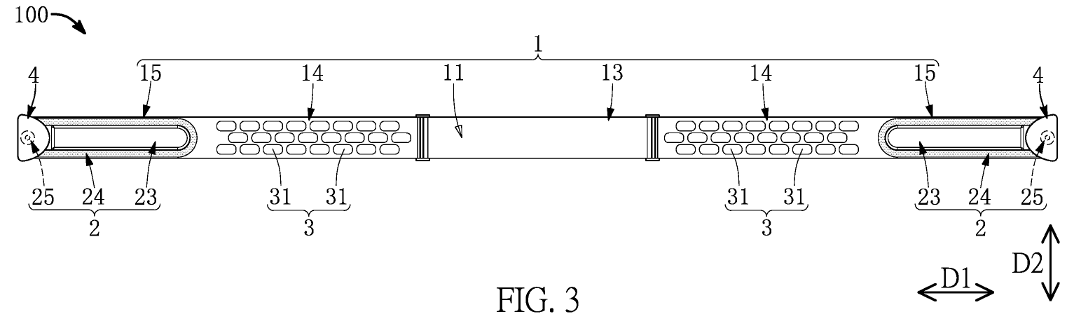

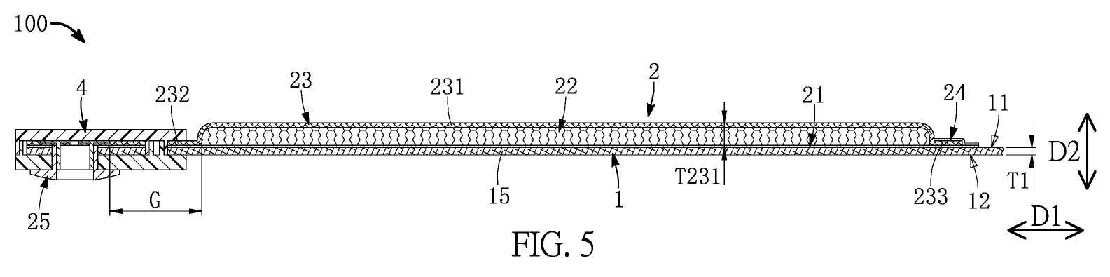

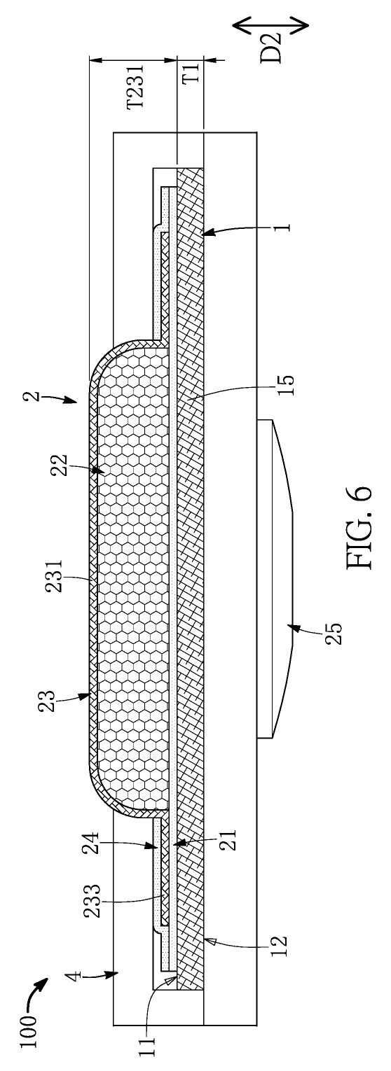

Referring to FIG. 1 to FIG. 7, a first embodiment of the present disclosure is provided. As shown in FIG. 1 to FIG. 3, the present embodiment provides a physiologic detection apparatus 1000, which includes a signal detection device 100 and a signal transceiver 200 that is detachably connected to the signal detection device 100. It should be noted that the signal detection device 100 in the present embodiment is described in cooperation with the signal transceiver 200, but the present disclosure is not limited thereto. For example, in other embodiments of the present disclosure not shown in the drawings, the signal detection device 100 can be independently used (e.g., sold) or can be used in cooperation with other components.

As shown in FIG. 1 and FIG. 4 to FIG. 6, the signal detection device 100 in the present embodiment includes a wearable carrier 1, two detection modules 2 disposed on the wearable carrier 1, two anti-slip layers 3 disposed on the wearable carrier 1, and two shells 4 that are respectively assembled to the two detection modules 2. The wearable carrier 1 has a first surface 11 and a second surface 12 that is opposite to the first surface 11, and the two anti-slip layers 3 and the two detection modules 2 are preferably disposed on the first surface 11 of the wearable carrier 1.

The wearable carrier 1 is elongated, a longitudinal direction of the wearable carrier 1 is defined as a first direction D1, and a width direction of the wearable carrier 1 is defined as a second direction D2 that is perpendicular to the first direction D1. Moreover, the wearable carrier 1 includes an elastic adjustment segment 13, two functional segments 14 respectively connected to two ends of the elastic adjustment segment 13, and two detection segments 15 that are respectively connected to two ends of the two functional segments 14 arranged away from each other. In other words, the elastic adjustment segment 13 and each of the two detection segments 15 are provided with one of the two functional segments 14 therebetween.

Specifically, the elastic adjustment segment 13 is configured to change or adjust a wearable length of the wearable carrier 1 for being applied to different wearing requirements. Each of the two functional segments 14 is provided with one of the two anti-slip layers 3 disposed thereon, each of the two detection segments 15 is provided with one of the two detection modules 2 disposed thereon and one of the two shells 4 that is assembled to an end thereof, and the signal transceiver 200 is detachably connected to the two detection modules 2 of the signal detection device 100, but the present disclosure is not limited thereto. For example, in other embodiments of the present disclosure not shown in the drawings, the wearable carrier 1 can be not elongated; or, a quantity of the detection modules 2, a quantity of the anti-slip layers 3, and a quantity of the shells 4 can each be one or more than one for being in cooperation with the wearable carrier 1 according to practical requirements.

It should be note that as the two detection modules 2 in the present embodiment are of substantially the same structure and are in a substantially mirror symmetrical arrangement, the following description discloses the structure of just one of the two detection modules 2 for the sake of brevity, but the present disclosure is not limited thereto. For example, in other embodiments of the present disclosure not shown in the drawings, the two detection modules 2 can be of different structures.

In the present embodiment, the detection module 2 includes a waterproof layer 21 disposed on the wearable carrier 1, a liquid storage layer 22 disposed on the waterproof layer 21, a conductive fabric 23 covering the liquid storage layer 22, a sealing layer 24 fixing a periphery of the conductive fabric 23 to the wearable carrier 1, and a signal transmission member 25 that is fixed to the conductive fabric 23 and the wearable carrier 1, but the present disclosure is not limited thereto.

The waterproof layer 21 is disposed on the first surface 11 of the wearable carrier 1 so as to enable the wearable carrier 1 to separate from the liquid storage layer 22 through the waterproof layer 21, thereby preventing liquid stored in the liquid storage layer 22 from flowing through the wearable carrier 1. The liquid storage layer 22 in the present embodiment is an absorbent soft material uniformly distributed along the first direction D1 (e.g., cotton of substantially equal thickness), but the present disclosure is not limited thereto.

The conductive fabric 23 is an elongated structure that is substantially parallel to the first direction D1. The conductive fabric 23 includes a contact portion 231, an extension portion 232 connected to the contact portion 231 along the first direction D1, and a fixing portion 233 that is connected to a periphery of the contact portion 231 and a periphery of the extension portion 232. The contact portion 231 covers the liquid storage layer 22 to enable the liquid storage layer 22 to be positioned between the contact portion 231 and the waterproof layer 21. The extension portion 232 is not in contact with the liquid storage layer 22 and extends from the contact portion 231 to a free end of the wearable carrier 1.

The signal transmission member 25 in the present embodiment is a rivet button, the signal transmission member 25 pierces through and is fixed to the wearable carrier 1 and the extension portion 232, and the shell 4 is fixed to the free end of the wearable carrier 1 and the signal transmission member 25. The signal transmission member 25 is spaced apart from the liquid storage layer 22 by a predetermined spacing G that is within a range from 5 mm to 12 mm, so that when the contact portion 231 is in a moist state by being in contact with the liquid storage layer 22, the contact portion 231 enables the extension portion 232 corresponding in position to the predetermined spacing G to be wetted through capillary action, thereby facilitating a signal transmission of the extension portion 232 and effectively preventing a transmission quality of the signal transmission member 25 from being affected by liquid.

The sealing layer 24 is formed on the first surface 11 of the wearable carrier 1 and the fixing portion 233 of the conductive fabric 23, such that the contact portion 231 is pressed to have a convex shape, and a distance between the contact portion 231 and the first surface 11 gradually decreases in a direction toward the fixing portion 233. Furthermore, a maximum distance T231 between the contact portion 231 and the first surface 11 is within a range from 150% to 250% of a thickness T1 of the wearable carrier 1, but the present disclosure is not limited thereto.

Accordingly, the signal detection device 100 is provided with the contact portion 231 having the convex shape and a specific size, thereby enabling the contact portion 231 to stably contact a to-be-detected portion of the user. Moreover, the wearable carrier 1 of the signal detection device 100 and the user have a gap therebetween through the contact portion 231, such that liquid (e.g., sweat) from the user can flow into the contact portion 231 by passing through the gap for being stored in the liquid storage layer 22.

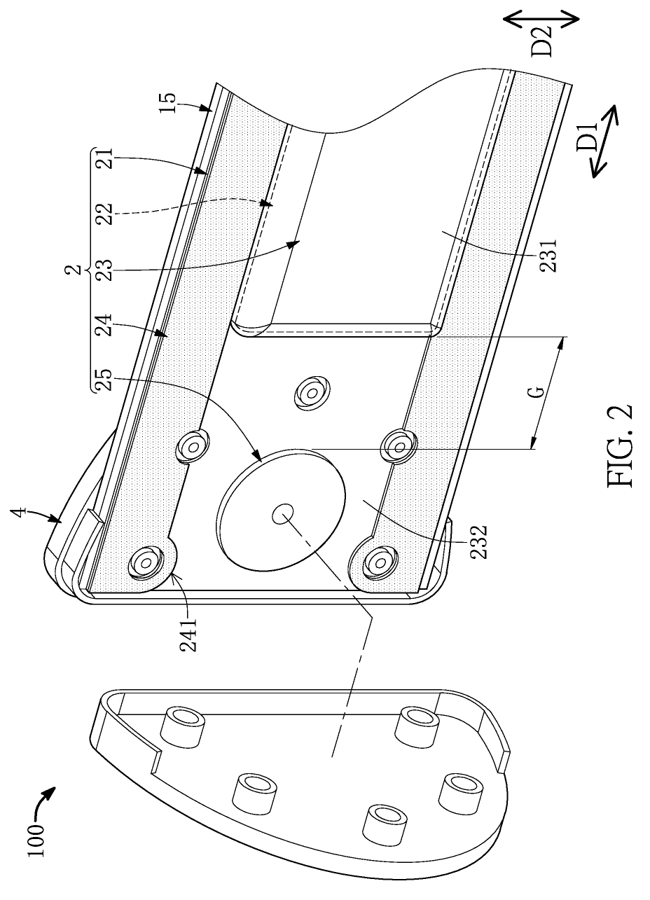

In the present embodiment, the sealing layer 24 surrounds at an outside of the liquid storage layer 22, an outside of the conductive fabric 23, and an outside of the signal transmission member 25. Specifically, the sealing layer 24 is U-shaped and has a notch 241 (shown in FIG. 2), the signal transmission member 25 is arranged adjacent to the notch 241, and the notch 241 is entirely covered by the shell 4.

The anti-slip layer 3 is disposed on the first surface 11 of the wearable carrier 1 and is spaced apart from the conductive fabric 23. In other words, the extension portion 232, the contact portion 231, and the anti-slip layer 3 in the present embodiment are arranged along the first direction D1. Accordingly, the signal detection device 100 is provided with the anti-slip layer 3, so that when a large amount of liquid exists between the wearable carrier 1 and the user, the anti-slip layer 3 is configured to effectively prevent the signal detection device 100 from sliding relative to the user.

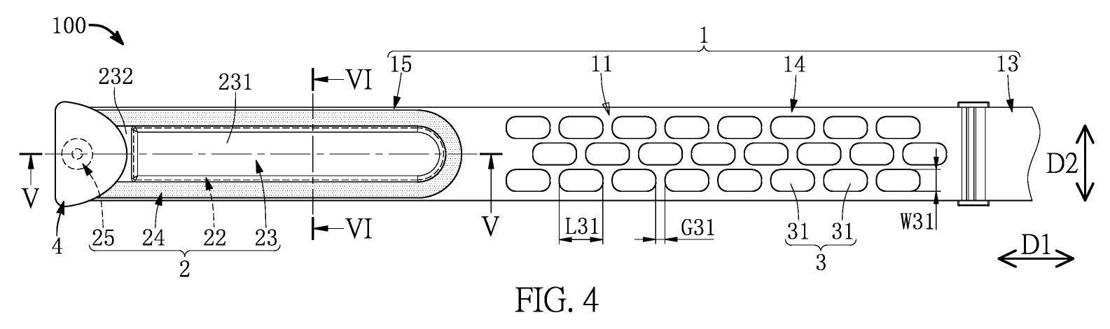

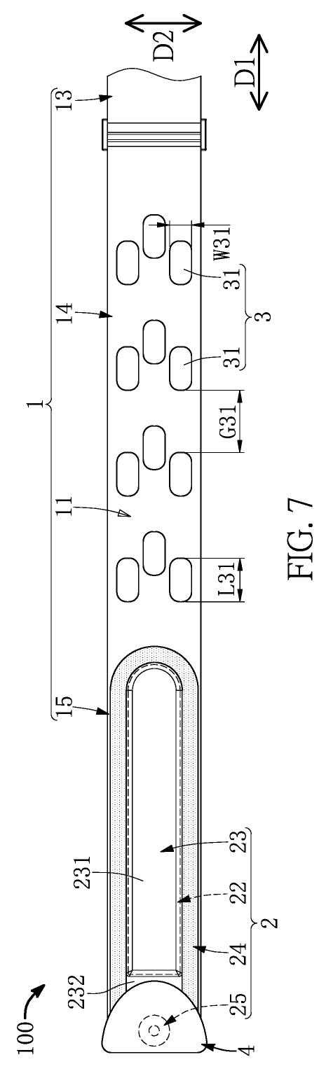

Specifically, in order to enable the anti-slip layer 3 to have a good anti-slip effect, the anti-slip layer 3 preferably has at least part of the following features, but the present disclosure is not limited thereto. The anti-slip layer 3 includes a plurality of anti-slip pads 31 spaced apart from each other. Each of the anti-slip pads 31 has a length L31 along the first direction D1 and a width W31 along the second direction D2, and the width W31 is less than the length L31. In any two of the anti-slip pads 31 arranged adjacent to each other along the second direction D2, one of the any two of the anti-slip pads 31 overlaps with an overlapped part of another one of the any two of the anti-slip pads 31 along the second direction D2, and a scale of the overlapped part is within a range from 25% to 50% of the length L31. In addition, along the first direction D1, any two of the anti-slip pads 31 arranged adjacent to each other are preferably spaced apart from each other by a layout distance G31 that is within a range from 0.5 mm to 3 mm. However, a distance between any two of the anti-slip pads 31 arranged adjacent to each other along the first direction D1 can be adjusted according to design requirements (as shown in FIG. 4 or FIG. 7).

In summary, the signal transceiver 200 is detachably connected to the signal transmission members 25 of the signal detection device 100 (i.e., the signal transceiver 200 is detachably connected to the signal transmission members 25 of the two detection modules 2 of the signal detection device 100). When the physiologic detection apparatus 100 is worn by the user, the contact portion 231 of the conductive fabric 23 is configured to receive a physiologic signal from the user, and the extension portion 232 and the signal transmission member 25 are configured to transmit the physiologic signal from the contact portion 231 to the signal transceiver 200.

### Second Embodiment

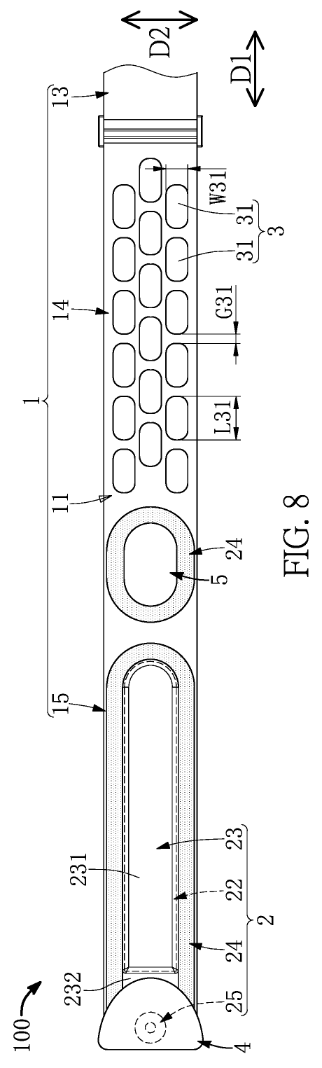

Referring to FIG. 8, a second embodiment of the present disclosure, which is similar to the first embodiment of the present disclosure, is provided. For the sake of brevity, descriptions of the same components in the first and second embodiments of the present disclosure will be omitted herein, and the following description only discloses different features between the first and second embodiments.

In the present embodiment, the signal detection device 100 further includes an antistatic layer 5 that is formed on the first surface 11 and that is arranged between the anti-slip layer 3 and the contact portion 231 of the conductive fabric 23. In other words, the anti-slip layer 3, the antistatic layer 5, and the conductive fabric 23 are arranged along the first direction D1. In addition, each of the two functional segments 14 of the wearable carrier 1 in the signal detection device 100 is provided with the antistatic layer 5 disposed thereon, but present disclosure is not limited thereto.

Accordingly, the signal detection device 100 in the present embodiment is provided with the antistatic layer 5, such that an accuracy and a stability of detection of the signal detection device 100 can be increased for meeting more requirements.

### Third Embodiment

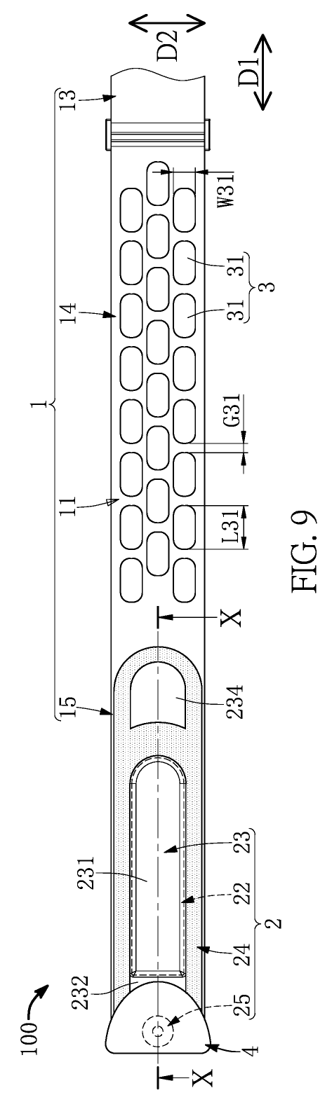

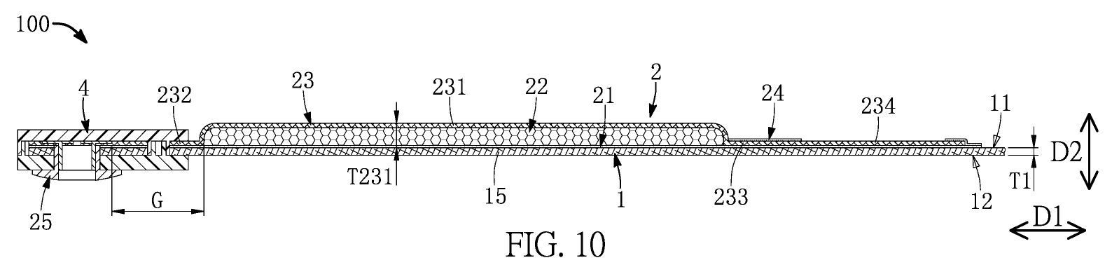

Referring to FIG. 9 and FIG. 10, a third embodiment of the present disclosure, which is similar to the first embodiment of the present disclosure, is provided. For the sake of brevity, descriptions of the same components in the first and third embodiments of the present disclosure will be omitted herein, and the following description only discloses different features between the first and third embodiments.

In the present embodiment, the conductive fabric 23 further includes an antistatic portion 234 that extends from the contact portion 231 in a direction away from the extension portion 232. The antistatic portion 234 is disposed on the first surface 11 and is not in contact with the liquid storage layer 22. Moreover, a periphery of the antistatic portion 234 is fixed to the wearable carrier 1 through the sealing layer 24. In addition, the antistatic portion 234 in the signal detection device 100 is formed by extending from the conductive fabric 23, but the present disclosure is not limited thereto.

### Beneficial Effects of the Embodiments

In conclusion, the physiologic detection apparatus and the signal detection device in the present disclosure are each provided with a structural cooperation among the wearable carrier, the liquid storage layer, and the signal transmission member (e.g., the extension portion extends from the contact portion and is not in contact with the liquid storage layer; and the signal transmission member is spaced apart from the liquid storage layer by a predetermined spacing that is within a range from 5 mm to 12 mm), so that when the contact portion is in a moist state by being in contact with the liquid storage layer, the contact portion enables the extension portion corresponding in position to the predetermined spacing to be wetted through capillary action, thereby facilitating a signal transmission of the extension portion and effectively preventing a transmission quality of the signal transmission member from being affected by liquid.

Moreover, the physiologic detection apparatus and the signal detection device in the present disclosure are each provided with the contact portion having the convex shape and a specific size (e.g., the maximum distance between the contact portion and the first surface being within a range from 150% to 250% of the thickness of the wearable carrier), thereby enabling the contact portion to stably contact a to-be-detected portion of the user. Moreover, the wearable carrier of the signal detection device and the user have a gap therebetween through the contact portion, such that liquid (e.g., sweat) from the user can flow into the contact portion by passing through the gap for being stored in the liquid storage layer.

In addition, the physiologic detection apparatus and the signal detection device in the present disclosure are each provided with the anti-slip layer, so that when a lot of liquid exists between the wearable carrier and the user, the anti-slip layer is configured to effectively prevent the signal detection device from sliding relative to the user.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A physiologic detection apparatus, comprising:
a signal detection device including:
a wearable carrier having a first surface and a second surface that is opposite to the first surface;
a waterproof layer disposed on the first surface of the wearable carrier;
a liquid storage layer that is disposed on the waterproof layer so as to enable the wearable carrier to separate from the liquid storage layer through the waterproof layer;
a conductive fabric including:
a contact portion that covers the liquid storage layer to enable the liquid storage layer to be positioned between the contact portion and the waterproof layer, wherein the contact portion has a convex shape, and a maximum distance between the contact portion and the first surface is within a range from 150% to 250% of a thickness of the wearable carrier; and
an extension portion connected to the contact portion and being not in contact with the liquid storage layer;
an anti-slip layer disposed on the first surface of the wearable carrier and spaced apart from the conductive fabric; and
a signal transmission member piercing through and being fixed to the wearable carrier and the extension portion, wherein the signal transmission member is spaced apart from the liquid storage layer by a predetermined spacing that is within a range from 5 mm to 12 mm;
wherein, when the contact portion is in a moist state by being in contact with the liquid storage layer, the contact portion enables the extension portion corresponding in position to the predetermined spacing to be wetted through capillary action; and
a signal transceiver detachably connected to the signal transmission member of the signal detection device, wherein, when the physiologic detection apparatus is worn by a user, the contact portion of the conductive fabric is configured to receive a physiologic signal from the user, and the extension portion and the signal transmission member are configured to transmit the physiologic signal from the contact portion to the signal transceiver.

2. The physiologic detection apparatus according to claim 1, wherein the conductive fabric includes a fixing portion that is connected to a periphery of the contact portion and a periphery of the extension portion, and a distance between the contact portion and the first surface gradually decreases in a direction toward the fixing portion.

3. The physiologic detection apparatus according to claim 1, wherein the signal detection device includes a sealing layer that is formed on the first surface and that fixes a periphery of the conductive fabric to the wearable carrier, and wherein the sealing layer surrounds at an outside of the liquid storage layer, an outside of the conductive fabric, and an outside of the signal transmission member.

4. The physiologic detection apparatus according to claim 3, wherein the sealing layer is U-shaped and has a notch, and the signal transmission member is arranged adjacent to the notch.

5. The physiologic detection apparatus according to claim 1, wherein the extension portion, the contact portion, and the anti-slip layer are arranged along a first direction, and the anti-slip layer includes a plurality of anti-slip pads spaced apart from each other and each having a length along the first direction, wherein, in any two of the anti-slip pads arranged adjacent to each other along a second direction perpendicular to the first direction, one of the any two of the anti-slip pads overlaps with an overlapped part of another one of the any two of the anti-slip pads along the second direction, and wherein a scale of the overlapped part is within a range from 25% to 50% of the length.

6. The physiologic detection apparatus according to claim 5, wherein, along the first direction, any two of the anti-slip pads arranged adjacent to each other are spaced apart from each other by a layout distance that is within a range from 0.5 mm to 3 mm.

7. The physiologic detection apparatus according to claim 1, wherein the signal detection device includes an antistatic layer that is formed on the first surface and that is arranged between the anti-slip layer and the contact portion of the conductive fabric.

8. The physiologic detection apparatus according to claim 1, wherein the conductive fabric includes an antistatic portion that extends from the contact portion in a direction away from the extension portion, and wherein the antistatic portion is disposed on the first surface and is not in contact with the liquid storage layer.

9. The physiologic detection apparatus according to claim 1, wherein the waterproof layer, the liquid storage layer, the conductive fabric, and the signal transmission member are jointly defined as a detection module, each of a quantity of the detection module and a quantity of the anti-slip layer is two, and the wearable carrier is elongated and includes:
an elastic adjustment segment configured to change a wearable length of the wearable carrier;
two functional segments respectively connected to two ends of the elastic adjustment segment, wherein each of the two functional segments is provided with one of the two anti-slip layers disposed thereon; and
two detection segments respectively connected to two ends of the two functional segments arranged away from each other, wherein each of the two detection segments is provided with one of the two detection modules disposed thereon;
wherein the signal transceiver is detachably connected to the signal transmission members of the two detection modules of the signal detection device.

10. A signal detection device, comprising:
a wearable carrier having a first surface and a second surface that is opposite to the first surface;
a waterproof layer disposed on the first surface of the wearable carrier;
a liquid storage layer that is disposed on the waterproof layer so as to enable the wearable carrier to separate from the liquid storage layer through the waterproof layer;
a conductive fabric including:
a contact portion that covers the liquid storage layer to enable the liquid storage layer to be positioned between the contact portion and the waterproof layer, wherein the contact portion has a convex shape, and a maximum distance between the contact portion and the first surface is within a range from 150% to 250% of a thickness of the wearable carrier; and
an extension portion connected to the contact portion and being not in contact with the liquid storage layer;
an anti-slip layer disposed on the first surface of the wearable carrier and spaced apart from the conductive fabric; and
a signal transmission member piercing through and being fixed to the wearable carrier and the extension portion, wherein the signal transmission member is spaced apart from the liquid storage layer by a predetermined spacing that is within a range from 5 mm to 12 mm;
wherein, when the contact portion is in a moist state by being in contact with the liquid storage layer, the contact portion enables the extension portion corresponding in position to the predetermined spacing to be wetted through capillary action.
